(19) United States
(12) Patent Application Publication
(10) Pub. No.: US 2016/0244547 A1
(43) Pub. Date: Aug. 25, 2016

(54) EFFICIENT COLLECTION OF NANOPARTICLES

(71) Applicant: NEW YORK UNIVERSITY, New York, NY (US)

(72) Inventors: Ramesh Jagannathan, New York, NY (US); Sachin Khapli, Mussaffah (AE)

(73) Assignee: New York University, New York, NY (US)

(21) Appl. No.: 15/027,657

(22) PCT Filed: Aug. 15, 2014
(86) PCT No.: PCT/US14/51272

§ 371 Date: Apr. 6, 2016

Related U.S. Application Data
(60) Provisional application No. 61/889,275, filed on Oct. 10, 2013.

(51) Int. Cl. C08F 224/00; B01J 3/04; B01J 3/00; A61K 31/192; A61K 9/51

(52) U.S. Cl. CPC C08F 224/00; A61K 31/192; A61K 9/5192; A61K 9/5146; B01J 3/008; B01J 3/04

(57) ABSTRACT

An improved processing technique that can be used to collect nanoparticles produced by the RESS process. The collection efficiency by almost an order of magnitude compared to the traditional collection processes. One process does not utilize any stabilizing solid co-solvents but produces similar effects using the supercritical solvent itself (e.g. CO₂) as a stabilizing phase. Very small particles (diameter <10 nm) with uniform size distribution and particulate suspensions thereof may be produced.

### CROSS REFERENCE TO RELATED APPLICATIONS

This application claims priority to U.S. Provisional Application No. 61/889,275 filed Oct. 10, 2013, reference of which is incorporated herein in its entirety.

### FIELD OF THE INVENTION

The present invention generally relates to synthesis and collection of small particles. Specifically, embodiments relate to collection of nanoparticles.

### BACKGROUND

Rapid expansion of supercritical solutions (RESS) is an attractive technique for the synthesis of nanoparticles with uniform size distribution. This method has attracted a great deal of attention in pharmaceutical applications where it is desirable to obtain sub-micron sized drug particles. In this method, a supercritical solution of a nonvolatile solute is allowed to expand through a small orifice resulting in rapid precipitation of the solute in the form of small, monodisperse particles. Because of the formation of extremely high super saturation the precipitation proceeds via homogeneous nucleation and results in ultra-fine particles. Indeed, the modeling studies of RESS process predict that nanoparticles in the size range 5-25 nm are formed at the onset of the fluid expansion zone. However, with the exception of a few reports, the final particle sizes reported are typically appreciably larger, around 500 nm to 10,000 nm (0.5-10 μm). In many applications, it is desirable to have the particle size much below 500 nm. For example, in pharmaceutical applications, sizes significantly less than 300 nm are preferred (Seki J, Sonoke S, Saheki A, et al. A nanometer lipid emulsion, lipid nano-sphere (LNS), as a parenteral drug carrier for passive drug targeting. Int J Pharm. 2004; 273:75-83.). In order to realize the full potential of the RESS technique, particle growth processes that occur downstream need to be minimized and an efficient way to recover the nanoparticles from the gaseous stream need to be devised.

Several methods have been proposed to suppress the particle growth by passivating the particle surface after their formation in the expanded medium. One of the methods, known as the rapid expansion of a supercritical solution into a liquid solvent (RESOLV), or RESS into an aqueous solution (RESS-AS), involves the collection of nanoparticles in a liquid medium, often containing surfactants and stabilizers that suppress their aggregation. Co-precipitation of the particles in the presence of passivating solutes has also been utilized, especially for creating composite particles. Recently, Gupta et al introduced a modified RESS process that overcomes the problem of particle aggregation in the expansion zone. In this process, known as the RESS-SC, a solid co-solvent (SC) namely menthol, is used. Besides increasing the solubility of organic compounds in the supercritical solution, it also suppresses the particle growth in the expansion zone by coating the particles.

There exist a number of applications where the form of the material is a limiting factor in usefulness. For example, within the pharmaceutical industry, it is well recognized that the phenomenon of poor solubility and hence reduced bioavailability has had a major negative impact on new drug development. This problem afflicts both the oral and parenteral drug delivery platforms. According to the Bio Classification System (BCS), under the Class 2 and 4 categories, majority of the New Chemical Entities suffer from poor water solubility and bioavailability. For certain Class 3 drugs, the problem is further compounded by poor solubility in organic media as well. The primary reason behind poor solubility leading to decreased bioavailability is the significantly decreased dissolution rates associated with these systems. Besides solubility, other key factors that impact dissolution rates of solid particles are their crystal habit and structure. For a chosen drug molecule, so far, there are few viable options to manipulate the crystal habit or structure of the “neat” solid drug particle. It is scientifically well established that solubility and hence dissolution rates of solid particles significantly increase with decreasing size. A precise control over drug particle size leads to more efficient targeted drug delivery and smaller particle sizes lead to higher rate of dissolution and enhanced rate of drug absorption. This, in turn, increases the bioavailability of the drug, smaller dosages and more controlled release. In the last decade or so, several researchers have been successful in creating nano-process platforms, labeled as micronization, that are capable of creating drug particles over a broad size range, without negatively impacting their bio-efficacy. Various traditional micronization strategies, viz. milling, grinding, crushing, freeze drying and etc., have been applied for particle size reduction of poorly aqueous soluble drugs. These traditional approaches involve numerous disadvantages such as thermal and chemical degradation, lack of level of control, large solvent consumption, solvent disposal issues, residual level of toxic solvents and broad particle size distribution. Similarly, the processing strategies applied for encapsulation of drugs within the polymer particles viz. phase separation, spray drying and double emulsion techniques, are beset with the same issues. Besides micronization, successful nano-process platforms for drug delivery could be broadly classified under synthetic (silica, polymers, gels) and natural (lipids, proteins, oligosaccharides). While many of these platforms bring unique advantages to the field of drug delivery, there still remains a certain attractiveness to a solution that does not require addition of other foreign molecules to the drug in the process. An ideal solution would be to remove the problem of dissolution rate entirely, by reducing their size to clusters of a few molecules, bound by weak, van der Waal's forces that would readily disassociate into molecules, during oral or parenteral drug delivery process. More particularly, development and collection of drug nanoparticles significantly less than 10 nm, with high yield has remained elusive, so far. Theoretical calculations were previously made by Helfgen et al. (B. Helfgen, P. Hils, Ch. Holzknecht, M. Turk, K. Schaber, Aerosol Science, 2001, 32, 295-319) modeling particle sizes in the range of 2-8 but in their set-up, expansion takes place through a capillary nozzle with a diameter of 60 μm and a length of 350 μm. The model compound is Cholesterol and the process conditions are: T=350-420 K and P=130 bar. The predicted particle size, 2-8 nm is only valid for the Mach disc of expansion and in the absence of particle growth processes downstream.

### SUMMARY

One implementation relates to a process comprising expanding a supercritical solution of a compound of interest through a capillary at supersonic speeds. The process further comprises creating a stream of nanoparticles dispersed in a gaseous medium. The stream of nanoparticles is cooled below the freezing point of the gaseous dispersion medium at least in part via application of external cooling provided by liquid coolant. The nanoparticles are captured in a solid matrix via a gas-to-solid phase change.

Another implementation relates to a nontransitory computer-readable memory having instructions thereon. The instructions comprise: instructions for expanding a supercritical solution of a compound of interest through a jet of nanoparticles particles dispersed in a gas; instructions for cooling the jet of ultra-fine particles below the sublimation point of the gaseous dispersion medium at least in part via application of external cooling provided by liquid coolant. The nanoparticles are separated are captured in a solid matrix via a gas-to-solid phase change.

Another implementation relates to a system for collecting nanoparticles. A feed system for provides a compound of interest. A feed cooling system having a pump, a cooling bath, a heat exchange is included. The feed cooling system provides supercritical solution of the compound of interest to a high pressure vessel. The high-pressure vessel is divided by a piston head, into a formulation chamber and a control chamber. A coolant vessel is in communication with the high-pressure vessel through a capillary tube.

Additional features, advantages, and embodiments of the present disclosure may be set forth from consideration of the following detailed description, drawings, and claims. Moreover, it is to be understood that both the foregoing summary of the present disclosure and the following detailed description are exemplary and intended to provide further explanation without further limiting the scope of the present disclosure claimed.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 shows the molecular structure of Teflon-AF

FIG. 2 is a schematic diagram illustrating the disclosed process.

FIG. 3 is a bar chart showing the comparison between various methods of capture of nanoparticles.

FIG. 4 is a SEM image of Teflon-AF nanoparticle samples obtained utilizing the described process.

FIG. 5 is an electron micrograph of a particle (˜10 nm) collected using an implementation of the invention.

FIG. 6 illustrates a computer system for use with certain implementations.

FIG. 7 is a histogram illustrating particle size and distribution for one implementation.

FIG. 8 illustrates instrumented Indentation Testing set-up (A) and Berkovich indenter (B).

FIG. 9 illustrates SEM micrographs of free nanosheets of drop casted emulsion Teflon-AF on TEM grid (A,B) and on silicon (C). Molecular stack of emulsion Teflon-AF thin film (D).

FIG. 10 illustrates XRD diffraction pattern of drop casted emulsion Teflon-AF thin film.

FIG. 11 illustrates AFM images of emulsified Teflon-AF drop casted on silicon substrate. Topography of tapping mode AFM (A,B) and height profile of molecular chain shown in B (C).

FIG. 12 illustrates load-displacement curves of different penetration depths. Low depth indentation of emulsion Teflon-AF thin film (A). High depth indentation of emuslion Teflon-AF thin film and uncoated silicon substrates (B).

FIG. 13 illustrates non-elastic behavior of Teflon-AF drop casted from emulsion (A). Elastic behavior of dropcasted Teflon-AF drop casted from Novec™ solution (B).

FIG. 14 illustrates low depth indentation of loading (A) and unloading (B).

FIG. 15 illustrates penetration depth of solution cast Teflon-AF thin film (A). Different sliding behavior of emulsion Teflon-AF thin film depending on loading time: 10 s (B), 20 s (C), and 30 s (D).

FIG. 16 illustrates Raman spectrum of emulsion-cast film of Teflon-A (A) and Raman spectrum of bulk Teflon-AF (B).

FIG. 17 illustrates AFM images of drop casted Teflon AF dissolved in Novec™. Height image (A) and phase image (B).

FIG. 18 illustrates Teflon-AF thin film drop casted from Novec™ solution (A) and from RESS emulsion solution (B).

FIG. 19 shows P-δ curves for various penetration depths of indented Teflon-AF drop casted films from Novec™ solution.

FIG. 20 shows a series of load and displacement (P-δ) curves of emulsion thin film carried out in 1 step (A), two steps (B), and four steps (C).

FIG. 21a is a graph of particle size distribution of RESS processed ibuprofen nanoparticles by dynamic light scattering (DLS);

FIGS. 22A-E relate to particle size distribution of RESS processed ibuprofen nanoparticles by Atomic force microscopy (AFM); FIG. 22A illustrates a AFM image of the nanoparticles, FIG. 22A-E are graphs of the size distributions at the four respective locations on FIG. 22A

FIG. 23 is a XRD spectra collected for Ibuprofen drug and processed nanoparticles

FIG. 24 is a graph of Ibuprofen measured yield percent at different extraction pressures.

### DETAILED DESCRIPTION OF THE PREFERRED EMBODIMENTS

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

In some implementations, systems and methods of the present invention are used in conjunction with production of nanoparticles. For example, one previously disclosed process for production of organic and organometallic nanoparticles with size between 2 and 25 nm is described in Ramesh Jagannathan, Glen Irvin, Thomas Blanton, and S. J. (2006). Organic Nanoparticles: Preparation, Self-Assembly, and Properties. Advanced Functional Materials, 16, 747-753 and Sadasivan; Sridhar, Jagannathan; Seshadri, Sunderrajan; Suresh, Merz; Gary E., Rueping; John E., Irvin, Jr.; Glen C., Jagannathan; Ramesh, Mehta; Rajesh V., Nelson; David J., U.S. Pat. No. 7,413,286, Method and apparatus for printing, Issued Aug. 19, 2008, both of which are incorporated by reference herein. As further described below, a method is provided to collect nanoparticles in high yield while minimizing the supplemental particle growth noted above regarding prior collection processes.

Certain implementations relate to a modified RESS process in which a similar effect to that achieved by a solid co-solvent is accomplished without the use of a solid co-solvent. A supersonic CO₂ jet containing nanoparticles is rapidly cooled below the sublimation temperature of CO₂ and the nanoparticles are captured in a solid matrix of dry-ice. In one implementation, the rate of cooling depends on the following variables: Operating conditions (i.e. Pressure and Temperature of sc-CO₂; these variables determine the magnitude of Joule-Thomson cooling that takes place during expansion), expansion geometry (diameter and length of the capillary tubing/nozzle; these variables affect the fluid dynamics of the expansion), and the rate heat transfer between the expanded CO₂ gas and the LN2 coolant. Formation of dry-ice coating on nanoparticles stops further particle growth. Formation of dry-ice coating on the surface of nanoparticles increases the drag and body forces acting on the particles, leading to their efficient capture from the gaseous stream. Thus, this method also enables efficient recovery of nanoparticles from the expanded fluid stream.

Kuo et al also describe a method of collection of nanoparticles in the RESS system using formation of dry ice. In the method of Kuo et al, an expansion tube is added after the nozzle to confine the expanded CO₂ gas and the internal Joule-Thomson cooling alone is utilized to freeze the gas upon expansion from supercritical solutions at pressure in the range of 345-1207 bar. The main problem with the Kuo method is that it entirely depends on the RESS (P,T) process parameters to induce the needed Joule-Thomson (JT) cooling to collect the nanoparticles in dry ice. In order to improve the collection efficiency, Kuo et al. direct the jet stream to impinge onto a collection container to form the dry ice. The nanoparticles formed by Kuo et al. are, like other prior art RESS processes, greater than 50 nm and often greater than 100 nm.

By comparison, implementations of the present invention remove the limitation of depending solely on the RESS system's JT cooling phenomenon to enable the dry ice formation by creating a separate cooling vessel. The use of a separate cooling vessel allows independent optimization of the cooling vessel to maximize the dry ice formation and collection efficiency. In one implementation this is accomplished by using “cold finger” vacuum traps and liquid N2 as coolant. In certain implementations, the external cooling vessel is capable of being scaled by adding several cooling vessels or several “cold finger” vacuum traps in series, to further optimize collection efficiency. Implementations of the present invention provide for collection of nanoparticles at significantly lower pressures than the process described by Kuo et al. Implementations of the present invention allow for the formation of smaller nanoparticles, less than 50 nm and in one implementation about 10 nm and in a further implementation, less than 10 nm.

As further noted below, in one implementation of the present invention a liquid nitrogen (LN₂) coolant to improve the freezing kinetics and also to increase the total amount of CO₂ frozen using considerably lower pressures (250-300 bar).

The formation and collection of nanoparticles using RESS with CO₂ includes a step wherein the solid CO₂ is formed. As noted, this relies upon the Joule-Thomson cooling. This presents several issues. First the formation of the solid CO₂ can be the rate-limiting step in the process. Thus, the faster the solid CO₂ forms, the faster and more efficient the overall process.

In one implementation, the use of an external cooling source overcomes the rate-limiting aspect of the formation of solid CO₂. The external cooling source allows for the formation of solid CO₂ at a rate significantly faster than would be possible with reliance up on J-T cooling alone. In effect, J-T cooling still occurs, but is supplemented by the use of the external cooling system. This allows for faster formation of solid CO₂.

A second issue with reliance on J-T cooling is the need for higher pressure in the system. This higher pressure and the reliance only on J-T cooling results in the formation of the solid CO₂ at the “throat” or throttling point where the pressure change occurs. This can result in clogging and is not a preferable location in the system for the formation of the solid.

In one implementation, the use of an external cooling source alters the location of the formation of the solid CO₂ or at least a portion of the solid CO₂, so as to avoid the formation of substantially all of the solid CO₂ at the “throat” or throttling point. Thus, the system and methods allow for more efficient processing with less down time to address clogging of the system.

A third issue that can occur in prior at J-T cooled systems is that the rate of cooling (hence solid CO₂ formation) is insufficient and the closed nature of the system, as no energy is removed, resulting in the reverse process of sublimation occurring. The formation of gaseous CO₂ from the already solidified CO₂ works contrary to the goal of the system and reduces yield and throughput.

In one implementation, the use of an external cooling source overcomes the inefficiencies caused by sublimation occurring within the system. The external cooling system results in faster cooling and more consistent cooling temperatures to reduce the occurrence of sublimation.

In one implementation, the use of an external cooling system allows for independent optimization of the cooling and the throttling. For example, the formulation pressure (and the consistency of a given pressure) impact the size of particles formed. The throttling within the system can be optimized without concern for the impact upon J-T cooling because cooling can be accomplished by the external cooling system. Likewise, the cooling can be optimized for a particular target compound that is being collected without concern for the impact on the throttling. For example, the prior art systems that relied only on J-T for cooling are limited in the variations that can be made to throttling, flow rate, and pressure due to the need to maximize conditions to ensure J-T cooling occurs.

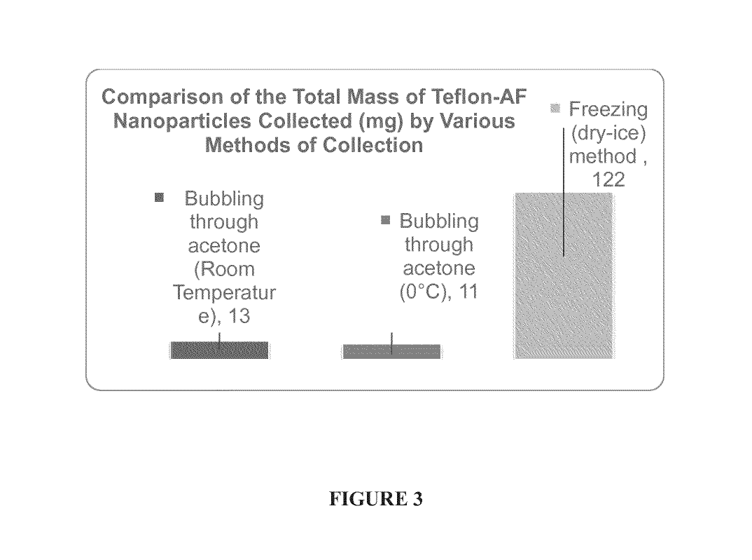

In one implementation, an improved processing technique can be used to collect nanoparticles produced by the RESS process. This implementation of the process improves the collection efficiency by almost an order of magnitude compared to the traditional collection processes. FIG. 3 illustrates a comparison of the nanoparticles collected. As can be seen, one implementation resulted in 122 mg of nanoparticles being collected, a nearly 10× increase over bubbling through acetone at room temperature and over a 10× increase over bubbling through acetone at 0 degrees Celsius. Unlike the RESS-SC process it does not utilize any stabilizing solid co-solvents but produces similar effects using the supercritical solvent itself (e.g. CO₂) as a stabilizing phase. It enables the production of very small particles (diameter <10 nm) and particulate suspensions thereof. In one implementation, surfactants are not used and are not necessary to stabilize the suspensions.

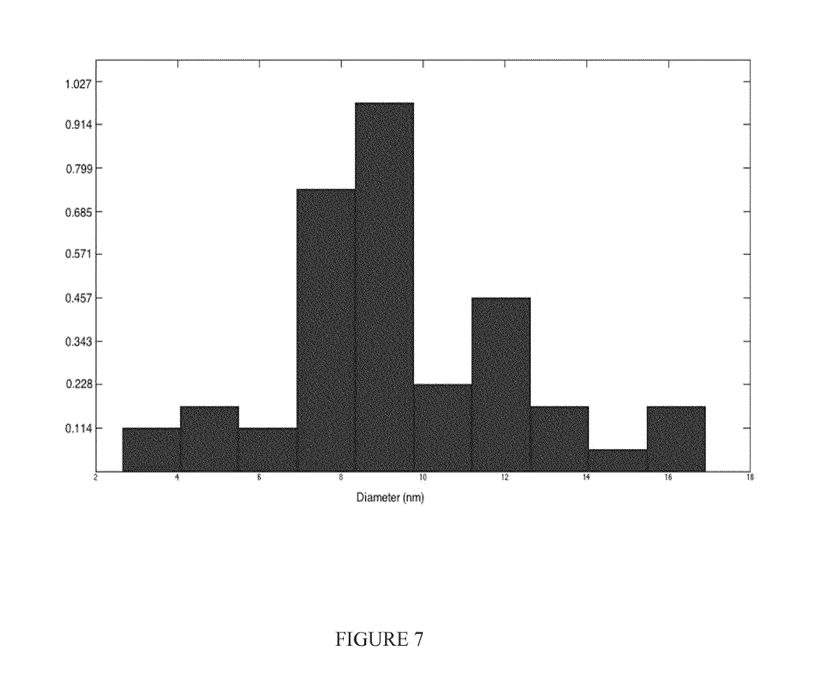

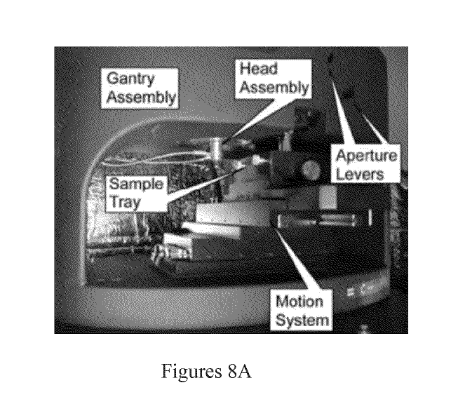

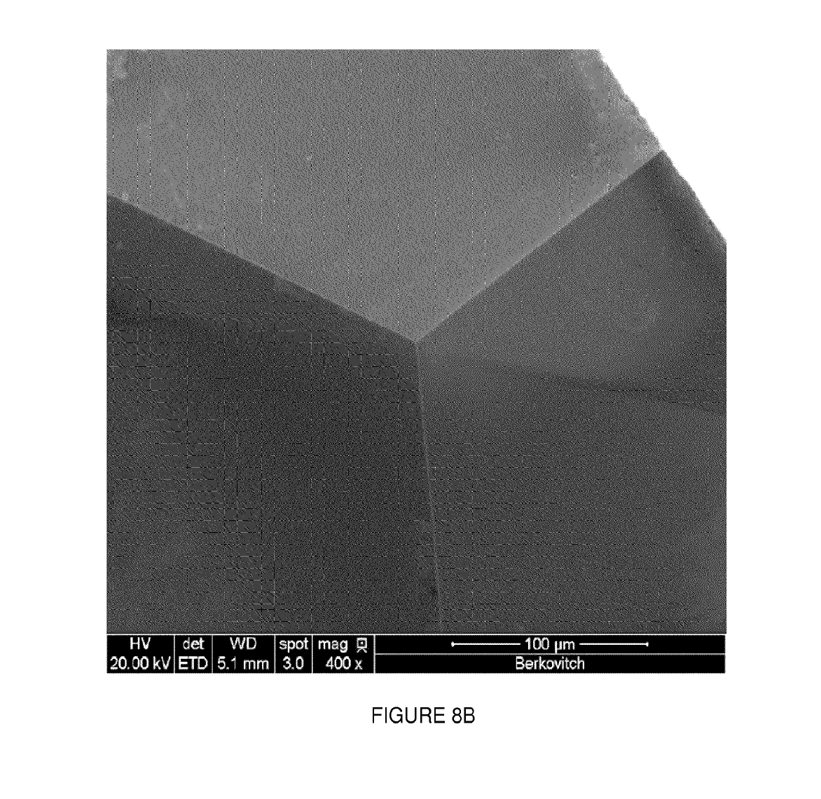

In one implementation, the particles created by the system and process described herein, have a narrower size distribution and a smaller average particle size than prior art systems. FIG. 7 illustrates a histogram of the particle size (x-axis in nanometers). As can be seen, the particles are primarily 10 nanometers and smaller. In addition, the percentage of particles captured by the solid CO₂, thus collected downstream is significantly increased. In one implementation, greater than 80% of the particles are captured in the solid CO₂. In another implementation, greater than 90% of the particles are captured in solid CO₂.

In one implementation, the system and method are applicable to nanoparticles, for example, but not limited to less than 20 nanometer to 1 nanometer. In a further implementation, the nanoparticles may be crystalline. In yet a further implementation, the particles may be encapsulated or coated. In one implementation, thermolabile substances may be used.

In one implementation, system and methods provide for no or no appreciable residual solvents, lower temperature processing, smaller particle sizes, and a “green” process through the recapture of the CO₂ from the process.

For one implementation, particle formation rates of 4 grams per hour have been observed.

Implementations can include the use of various delivery formats such as, but not limited to: uniform films, quick dissolve patches, inhalation systems, nanoinvasive injections, imbedded patterned tablets and capsules, transdermal materials, and nanodispersions via oral/injectibles.

In certain implementations, nanoparticles (e.g., 10-100 nm) can be collected in high yield and dispersed in either organic solvents (acetone, ethanol, and n-heptane) or at the air-water interface to create thin films. It is believed this is characteristic of polymeric systems (for example, the Teflon AF in the example below) rather than small molecules or molecular clusters under 10 nm.

Certain implementations related to systems and methods to collect nanoparticles are of interest in several pharmaceutical applications. Application of implementations that include processing of Teflon-AF also yields interesting morphologies of Teflon-AF that could be beneficial for several other existing applications. For example, the current methods of processing Teflon-AF using fluorinated solvents do not yield superhydrophobic films. The largest contact angle achieved by spin coating is 135° because of the absence of nanoscale roughness. However, at least one implementation of the present invention yields superhydrophobic surfaces with contact angles up to 162°. Film formation at the air-water interface or by drying of dispersion of Teflon AF nano particles causes the formation of porous films composed of nano particles. Such films are characterized by dual hierarchy of surface roughness (micro+nano) and low surface energy. This makes the surface superhydrophobic.

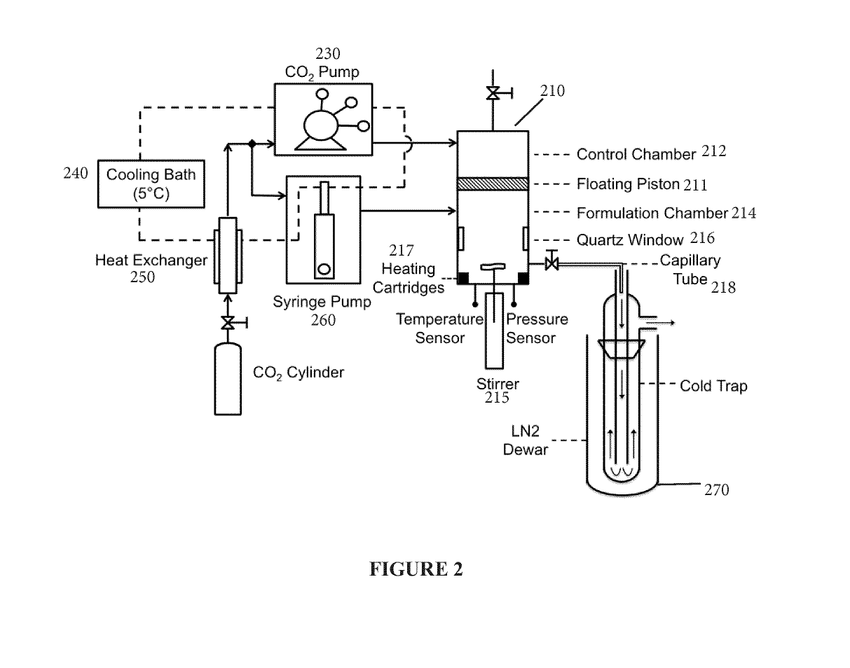

FIG. 2 shows a schematic of one implementation of an RESS. The includes a high-pressure vessel 210, which is divided by a piston head 211, in one implementation a gas-tight, floating piston head, into two compartments: the formulation chamber 214 and the control chamber 212. The system further includes a pump 230, a cooling bath 240, a heat exchanger 250 and a syringe pump 260. During an experiment, pressure inside the formulation-chamber 214 is maintained at a desired, constant value with the help of the piston head 211. The piston head 211 may be computer-controlled such that it is moved forward by controlled addition of compressed CO₂ to the control chamber 212. The formulation chamber 214 consists of a stirrer 215. The formulation chamber 214 may further include one or more viewing windows 216, such as two sapphire viewing windows, and the temperature inside it is maintained by computer-controlled cartridge heaters 217 inserted into the chamber walls. Temperature and or pressure sensors may be included in the high-pressure vessel 210. An external coolant vessel 270 is in communication with the pressure vessel. In one implementation, a capillary tube (for example 25-200 micron) connects the pressure vessel 210 with the coolant vessel 270.

In one implementation, the coolant vessel 270 is a vacuum trap (“cold finger” type) is used for collection of dry-ice because it offers a favorable geometry for condensation of the gaseous stream: the inner tube of the vacuum trap helps confine the flow and the outer surface provides a cryogenic interface for freezing the gaseous stream.

In one implementation, the process comprises rapidly expanding a supercritical solution of a compound of interest through a fine capillary, for example, 25-200 micron ID capillaries at supersonic speeds (i.e. the RESS process). In one implementation, the expansion takes place from pre-expansion pressure (100-300 bar) to atmospheric pressure through a capillary. The fluid velocity at the outlet ranges from 200-300 m/s and the corresponding residence time is 166-250 ms.

A jet of ultra-fine particles, in the size range of 2-200 nm is created, dispersed in a gas. The jet is immediately cooled to below the freezing point of the gaseous dispersion. For example, the cooling may be accomplished by utilizing the local cooling in the jet due to the Joule Thomson effect as well as external cooling provided by liquid nitrogen (LN₂) coolant so that a gas-to-solid phase transformation takes place in the jet and the nanoparticles get embedded in the solid matrix formed.

In one implementation, the CO₂ matrix may be stored, including intended for long-term storage of the particles within the CO₂ matrix. In such implementations, the higher efficiency of the collection system results in a much more efficient storage. For example, 10 times the amount of C02 matrix would be necessary to store the same amount of target compound particles in the prior art compared to the implementation of the present invention based upon the data shown in FIG. 3.

### EXAMPLES

### 1. Teflon-AF 1600

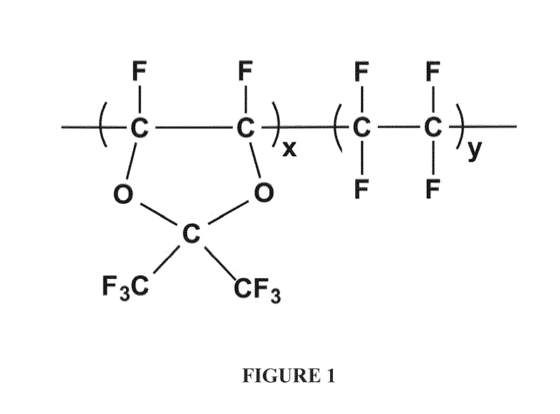

Materials: Teflon-AF 1600, was procured from the DuPont™ Corporation (Wilmington, Del., USA) in an amorphous resin form and used without further purification. Chemical structure of the copolymers belonging to the Teflon-AF family is shown in FIG. 1. Amorphous Teflon (Teflon-AF) is a class of fluoropolymers prepared by copolymerization of 4,5-difluoro-2,2-bis(trifluoromethyl)-1,3-dioxole (BDD) and tetrafluoroethylene (TFE) (FIG. 1). Designed at the molecular level, these materials are well known for their outstanding optical, electrical, and mechanical properties combined with excellent chemical inertness and biocompatibility. In these macromolecules, the introduction of bulky dioxole moieties to a PTFE backbone results in an amorphous structure with a large fractional free volume. The high free volume combined with the amorphous structure imparts better mechanical and optical properties to the material as compared to the microcrystalline Teflon polymer. Teflon-AF materials exhibit interesting mechanical properties such as high compressibility, high creep resistance to tensile and compressive loads, and low coefficient of friction. These properties combined with their excellent thermo-chemical resistance, low dielectric constant, and high gas permeability enable several applications of the material across the semiconductor, optoelectronic, and biomedical industries. Nanoscale coatings of Teflon-AF are used in waveguides, anti-reflective coatings, low-k dielectric films in semiconductor devices, and antifouling surfaces in microfluidic devices. Knowledge of the mechanical properties of Teflon-AF films at nanoscale spatial resolution is therefore necessary for the design of such devices. These are random copolymers of tetrafluoroethylene (TFE) and 2,2-bis-trifluoromethyl-4,5-difluoro-1,3-dioxole (PDD) and their physical properties exhibit a gradual variation with the mole fractions of the co-monomers. Teflon® AF copolymers are commercially available in two grades, AF-1600 and AF-2400, consisting of 64 and 83 mol % PDD, respectively, and characterized by glass transition temperatures of 160 and 240° C., respectively. Teflon AF 1600 with Mₙ˜100 kDa was used in the RESS experiments.

RESS Process: The system of FIG. 2 was used in the experiment. To facilitate the dissolution of compounds in sc-CO₂, the stirrer 215 is a high speed mixer (57 watts @ 2400 rpm) is provided inside the formulation chamber 214. A capillary tube 218, for example stainless steel with 125 μm internal diameter and 1 in. length, is attached to the chamber 214 such as via a calibrated, manual needle valve. The formulation chamber 214 is first loaded, such as with a weighed quantity of Teflon-AF 1600 (5 gm, solid powder) and sealed tightly. Liquid carbon dioxide (purity level 99.9%) is then injected into the chamber 214 by the syringe pump 260. The operating pressure and temperature are then set to the desired values, 250 bar and 60° C. respectively, enabling transition of carbon dioxide to a supercritical state. Volume of supercritical carbon dioxide added under these conditions is 216±4 mL. The system 200 is then allowed to equilibrate for 1 h with constant stirring so that the fluoropolymer dissolves completely in the supercritical fluid medium. Upon complete dissolution, nanoparticles of AF 1600 are precipitated by allowing the supercritical fluid to escape through the capillary tubing. The supersonic CO₂ jet exiting from the capillary is directed into a vacuum trap cooled below the sublimation point of solid carbon dioxide (T≦−78° C., coolant medium: liquid Nitrogen) with additional cooling provided by the Joule-Thomson effect. A mass of dry-ice forms in the vacuum trap, which contains the nanoparticles of Teflon AF embedded in the dry-ice matrix.

In one implementation, a vacuum trap (“cold finger” type) is used for collection of dry-ice because it offers a favorable geometry for condensation of the gaseous stream: the inner tube of the vacuum trap helps confine the flow and the outer surface provides a cryogenic interface for freezing the gaseous stream.

Calculation of Collection Efficiency of Nanoparticle Capture: In the described method, the supersonic CO₂ jet containing nanoparticles is rapidly cooled below the sublimation temperature of CO₂ and the nanoparticles are captured in a solid matrix of dry-ice. In the absence of cooling, nanoparticles tend to be carried away in the gas stream. However, if the CO₂ jet containing nanoparticles is cooled to temperatures below 195 K, nanoparticles are encapsulated inside dry ice which prevents their aggregation. Encapsulation of nanoparticles with dry-ice also increases the body forces on the particles and results in their deposition inside the container. Overall, this leads to an increase in the amount of nanoparticles collected from the effluent CO₂ jet.

The increase in efficiency of capture of nanoparticles in this modified method can be calculated as follows: In the described apparatus (FIG. 2) the total volume of formulation chamber is a function of the position of the piston and therefore one can measure the total volume of sc-CO₂ expanded by tracking the displacement of the piston during the expansion process. The mass of dry-ice formed due to Joule-Thomson effect can be measured by weighing it in a tightly sealed vial. The mass of nanoparticles embedded in dry-ice is negligible compared to that of the dry-ice but can be measured after gravimetric analysis of the dispersion obtained by dissolving the dry-ice in acetone. In a typical experiment, 50 ml of sc-CO₂ undergoing expansion yields approximately 20±2 gm of dry ice containing Teflon AF nanoparticles. Since the density of Sc—CO₂ under these conditions is 0.775 g/ml, this implies that 51.6% of the exiting stream gets condensed with a non-optimized set up (An optimized set up would maximize the CO₂ condensation rate by improving heat transfer rate between the exiting gaseous stream and the nanoparticle collection chamber). When the entire volume of sc-CO₂ enclosed in the reaction chamber is expanded and the nanoparticles collected in dry ice, the total mass of nanoparticles collected is 122±47 mg.

For determining the efficiency of the method of collection, two separate control experiments were carried out. In one experiment, where the gaseous stream is bubbled through acetone solvent at 0° C., the same piston displacement results in collection of approximately 11±8 mg of Teflon AF nanoparticles. In another experiment, where the gaseous stream is bubbled through acetone solvent at room temperature, the same piston displacement results in collection of approximately 13±6 mg of Teflon AF nanoparticles. Therefore, the method of capture results in nearly an order of magnitude increase in the collection efficiency (FIG. 3). Dry-ice with embedded nanoparticles can be stored in a cryo-freezer at 77 K for long term storage or dissolved in various organic solvents to obtain surfactant-free dispersions of nanoparticles.

Formation of surfactant-free dispersions: Small chunks of dry-ice containing Teflon-AF nanoparticles are mixed with suitable organic solvent (acetone, ethanol, and n-heptane) and allowed to thaw in an open container. Upon complete sublimation of dry-ice, surfactant-free dispersions of Teflon-AF are formed. Films are cast by drop casting of the dispersions on silicon substrates.

Formation of free-floating film on water: Small chunks of dry-ice containing Teflon-AF nanoparticles are allowed to thaw over de-ionized water in a glass beaker. Chunks of dry-ice are instantly coated with frozen water upon dropping in de-ionized water. Upon complete sublimation of dry-ice, a semi-transparent film is seen floating on the water surface which is easily transferred onto silicon substrates by dip-coating.

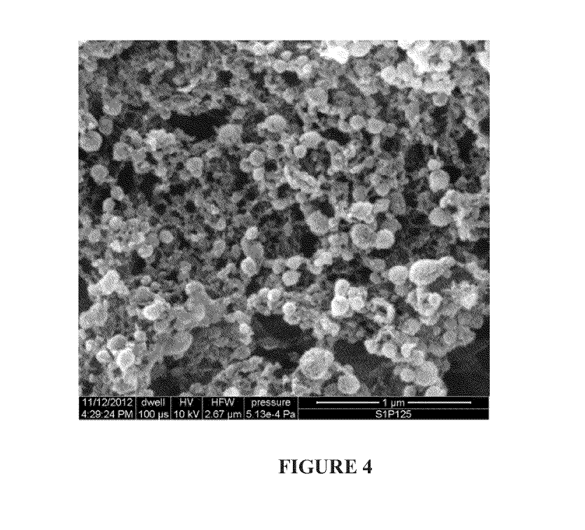

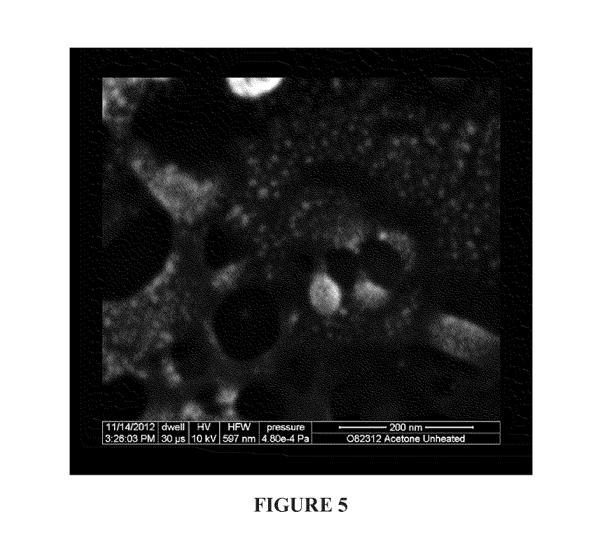

SEM imaging of Teflon-AF films: Teflon-AF films are cast from surfactant-free dispersions in acetone by drop-casting at 60° C. on silicon wafers and their morphology is studied by SEM. As shown in FIG. 4, the films are porous and consist of a membrane-like network of polymer nanoparticles. The porosity of the films could be due to the formation of weakly bound, fractal aggregates of the smaller, primary nanoparticles formed by his process, which are shown in FIG. 5, during the evaporation of the solvent.

a. Nanoindentation Characterization

Teflon-AF can be further characterized to determine the nanoscale and microscale structure As noted above, knowledge of the mechanical properties of Teflon-AF films at nanoscale spatial resolution is therefore necessary for the utilization of the material in specific applications.

The technique of nanoindentation has been well developed over the past decade and applied to the mechanical characterization of thin films with thickness in the nanoscale regime. In this method, an indenter is allowed to make contact with the surface of interest and pressure is gradually applied while monitoring the depth of penetration. From the load-displacement (P-δ) curves it is possible to determine several mechanical properties of interest such as the hardness, elastic modulus, yield stress, and viscoelastic properties for thin films immobilized on a rigid substrate. The method has been applied for measurement of thin-film properties of metallic, ceramic, semiconductor, and polymeric materials, with international standard procedures (ISO 14577) being applied for analysis. Nanoindentation measurements of ultrathin polymeric films are complicated by several factors such as the range of soft loads required, viscoelastic/plastic nature of the indentation response, anisotropic nature of the properties due to polymer microstructure and continue to be an active area of research.

The Teflon-AF produced using an implementation of the above described RESS technique are compared to prioar films obtained by processing of Teflon-AF in a fluorinated solvent, Novec™ 7100 (i.e. methoxy-nonafluorobutane). Films obtained by the latter method are not superhydrophobic and lack the surface roughness and porous microstructure observed in the films prepared by supercritical fluids based processing of Teflon-AF.

i. Experimental:

Materials: Teflon®-AF 1600, was procured from the DuPont™ Corporation (Wilmington, Del., USA) in an amorphous resin form and used without further purification. Novec™ HFE 7100 (methoxy-nonafluorobutane) was obtained from 3M Specialty Materials, St. Paul, Minn. It consists of a mixture of isomers of methoxy-nonafluorobutane in unknown proportion. De-ionized water with an electrical resistance of 18.2 MΩ·cm was used to prepare all aqueous solutions. Liquid carbon dioxide with purity of 99.9% was used to create the supercritical fluid medium for dissolution. Silicon wafers ((100) orientation, polished, 380 μm prime grade) were purchased from University Wafers Inc. (South Boston, USA) and cut into 1 cm×1 cm pieces for sample preparation.

Methods: Formation of surfactant-free dispersions of Teflon-AF, formation of free-floating films on water, and solution processing of Teflon-AF in Novec™ HFE 7100 solvent were performed as described above. A free-floating Teflon-AF film formed on a water surface was sonicated after adding a cyclohexane layer on top (volume ratio of cyclohexane:water=1:10). Sonication was performed using a Branson 250 Sonifier (tub sonicator) for 30 mins at room temperature in a covered beaker. Films were formed on clean Si wafers by air-drying drop cast solutions of emulsion droplets at room temperature.

Optical and Raman microscopy was performed on a WiTec alpha 300 confocal Raman microscope equipped with 50× and 100× objectives. The pinhole diameter of the confocal microscope was kept constant at 100 μm. Raman spectra were acquired using 488 nm laser for excitation (10 mW power) and recorded using a CCD camera maintained at −60° C. Integration time for acquisition of spectra was kept constant at 1 second. Each spectrum is an average of 10 consecutive scans. Teflon-AF films were rescanned after data acquisition to detect radiation-induced damage. No chemical change in the film composition was detected for an exposure time of 10 seconds at 10 mW power. In addition, SEM images of Teflon-AF polymeric films immobilized on solid substrates were obtained using a FEI Quanta FEG 450 electron microscope (acquired at 5-20 kV accelerating voltage and 10⁻³−10⁻⁴ Pa pressure).

ii. Nano Indentation Experiments:

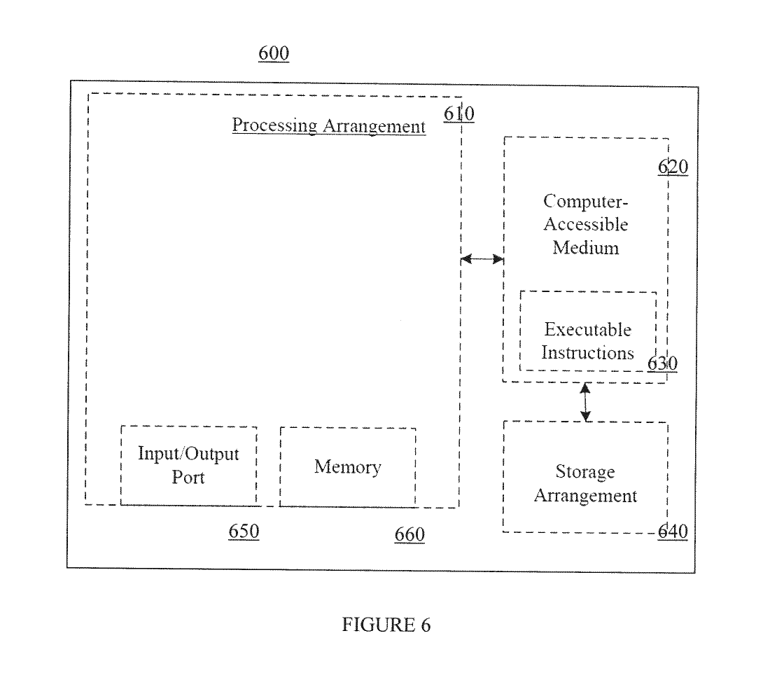

The nano indentation experiments were performed using an Agilent G200 Instrumented Indentation Testing (IIT) nanoindenter, equipped with a diamond Berkovich indenter with a radius less than 20 nm (FIG. 6). The system is in compliance with ISO 14577. Traditional hardness testing yields only one measure of deformation at one applied force, whereas during an IIT test, force and penetration are measured for the entire time that the indenter is in contact with the material. Instrumented indentation testing is particularly well suited for measuring Young's modulus (E) and hardness (H) of material such as thin films, particles, or other small features.

Two methods were used to conduct nanoindentation, namely, basic hardness testing with continuous load-displacement data and Continuous Stiffness Measurement (CSM). For the basic hardness, the indenter was loaded at a constant rate until reaching the specified peak load, hold for specific time, and then unloaded at the same rate. The steps for reaching the maximum load can also be specified during the tests. The CSM option allows the continuous measurement of the contact stiffness during loading and is not just at the point of initial unload. This was accomplished by superimposing a small oscillation on the primary loading signal and analyzing the resulting response of the system by means of a frequency-specific amplifier. With the continuous measure of contact stiffness, one obtains the hardness and elastic modulus as a continuous function of depth from a single indentation experiment.

The samples for both methods were mounted on aluminum disks using Crystalbond™, a thermoplastic polymer, and loaded to the sample tray capable of holding up to four samples at the same time. The nano indentation system was placed on a vibration isolation table and it is equipped with a 10× and 40× objectives making it possible to adjust the height of the samples using the reference sample, Corning 7980 (fused silica), located at the center of the sample tray. During each experimental batch, control experiments were run with a fused silica sample to ensure quality control, keep track of the performance of the instrument and indirectly verify proper operation according to ISO-14577. All experiments were conducted at room temperature.

The system specifications for the nanoindenter are as follows: displacement resolution: <0.01 nm, total indenter travel: 1.5 mm, maximum indentation depth: >500 μm, load application: coil/magnet assembly, displacement measurement: capacitance gauge, maximum load (standard): 500 mN, load resolution: (XP 50 nN, contact force: <1.0 μN, load frame stiffness: approximately 5×10⁶ N m⁻¹, and software: NanoSuite

iii. XRD Experiments:

Samples were measured using a Panalytical Empyrean diffraction system using Cu Kα radiation (1.5418 Å). The samples were irradiated using a parallel beam focusing mirror, 0.04 radian Soller slits, and a 0.27° parallel slit collimator placed before the scintillation detector. The samples were aligned for X-ray reflectivity, with the sample blocking half the incident intensity and symmetric reflection geometry (ω=θ). Step scan measurements were made with a step size of 0.01°2θ and a count time of 16 s step⁻¹.

AFM measurements were carried out in intermittent mode in air using Agilent MAC Mode III module. Silicon Point Probe Plus (PPP) cantilevers (Nanosensors, Switzerland) with a resonant frequency of 330 kHz and spring constant of 42 N m⁻¹ were used. Height, phase and amplitude images were acquired simultaneously. The images were further analyzed by using Gwyddion free software.

iv. Results and Discussions:

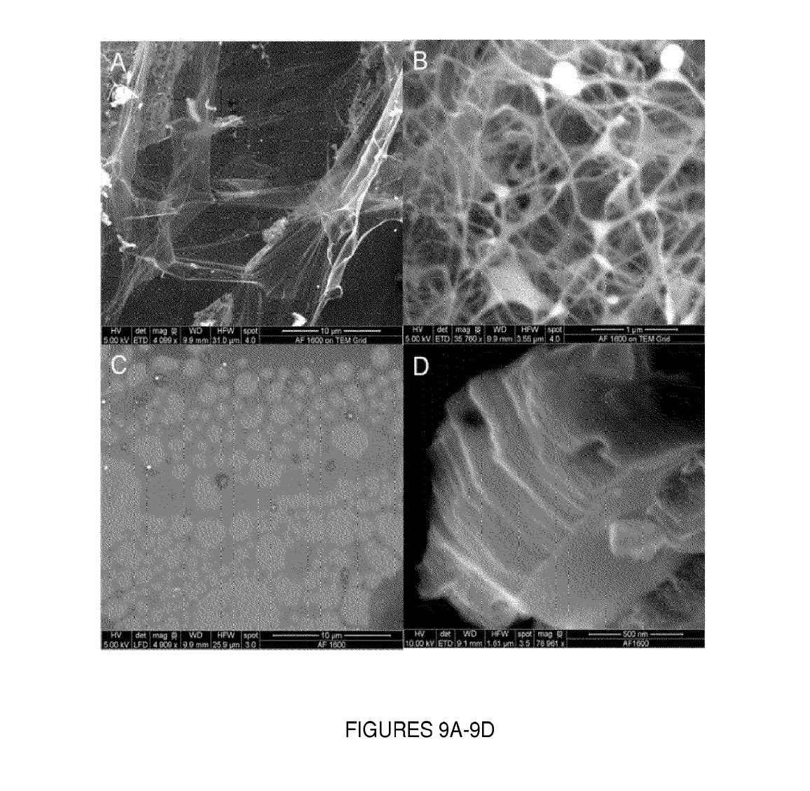

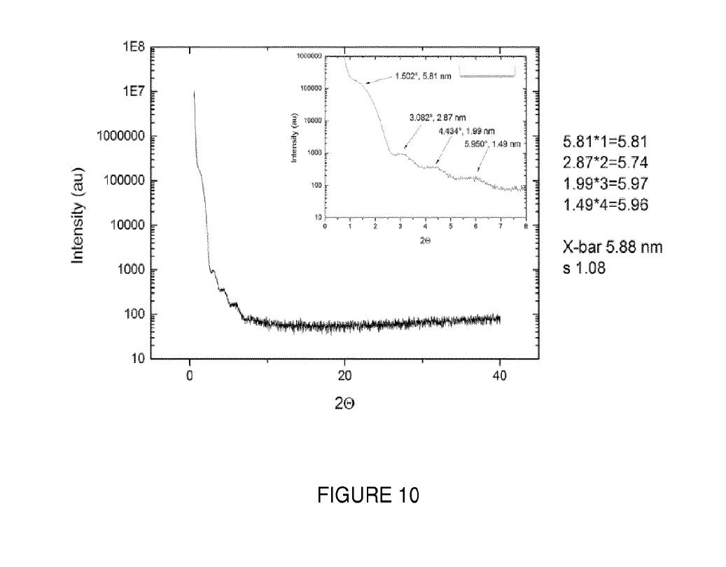

Freestanding films of Teflon-AF were created by the RESS process at the air-water interface. Electron micrographs (FIGS. 9A and 9B) of samples of this film drop cast on a TEM grid and dried at room temperature show evidence of mechanically robust, extremely thin sheets of Teflon-AF. An emulsification process was used to further increase the specific surface area of these films and the oleophilic nature of these films was expected to drive their spontaneous self-assembly at the oil-water interface. The electron micrographs of this film, drop-cast on a silicon substrate and dried at room temperature, is shown in FIG. 9C and appears to support the notion of film formation by the drying of coalesced emulsion droplets. At the oil-water interface, the surface tension forces are expected to further stretch the films resulting in the formation of stacked layers of nanosheets which are clearly evident in the electron micrograph shown in FIG. 9D. The image shown in FIG. 9D is from the same film sample shown in FIG. 9C but from an area where two pieces have broken off from the main film thereby exposing the sliced inner surface. Results from low angle XRD characterization of the same film sample are presented in FIG. 10. Low angle peaks at near equiangular Δ2θ are consistent with multiple order diffraction peaks (1^{st}, 2^{nd}, 3^{rd}, 4^{th} order) and combined with the SEM data in FIG. 9D an indication of the presence of a uniform thickness layered component. The absence of diffraction peaks at higher 20 angles confirms the amorphous nature of the Teflon-AF thin film. Calculation of the interplanar d-spacings for the low angle diffraction peaks multiplied by the order of the corresponding diffraction peak gives an average nanosheet layer thickness of 5.87±0.11 nm.

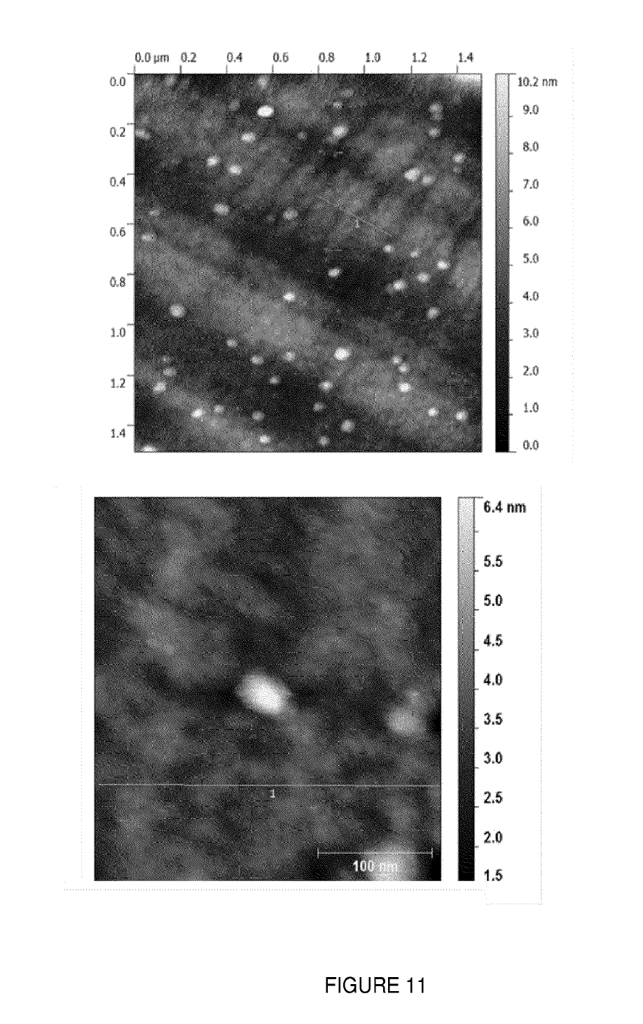

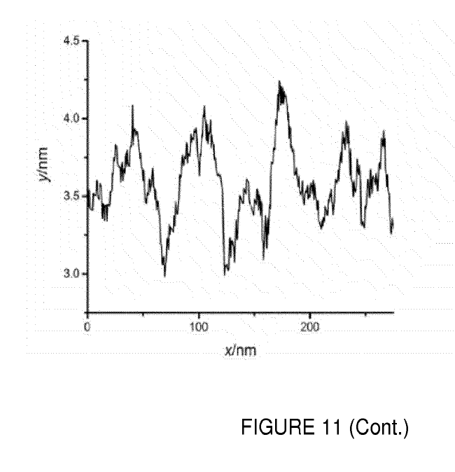

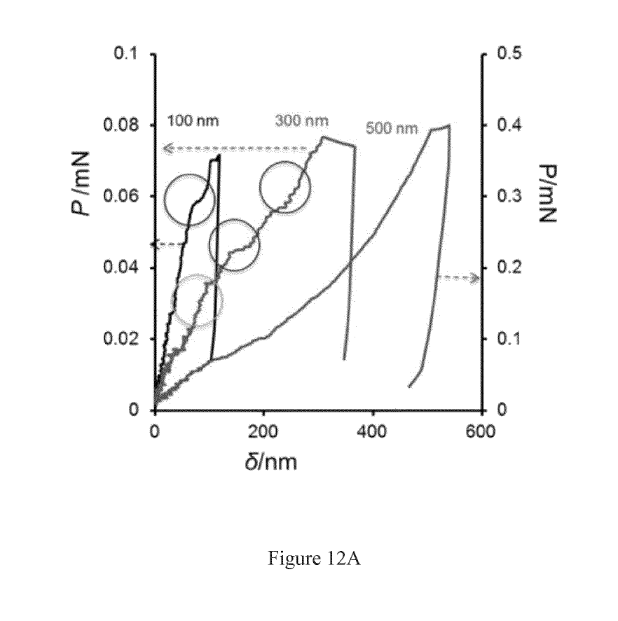

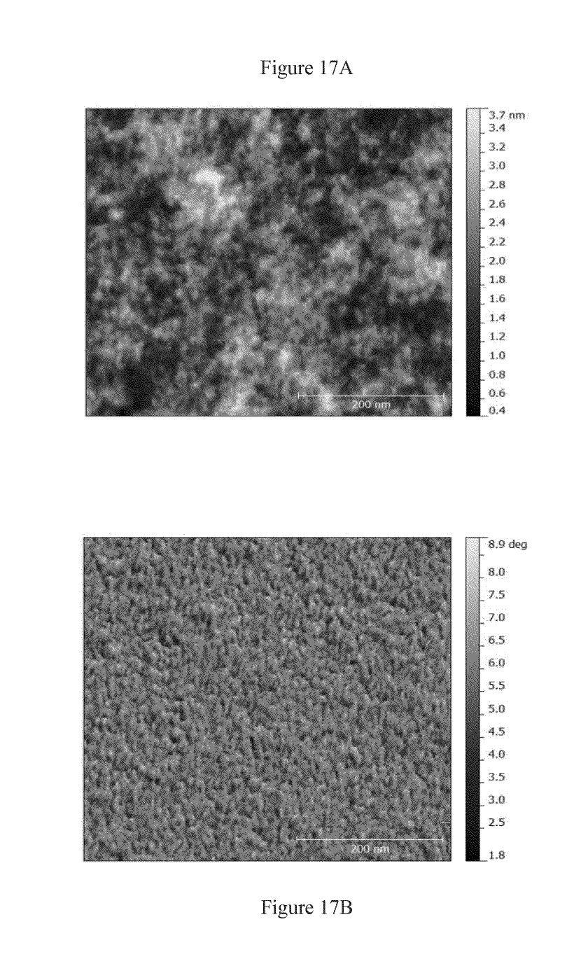

AFM examination of this film further validated the electron micrograph observations and the low angle XRD data. The results shown in FIGS. 11A and 11B indicate the presence of self-assembled, ordered arrays of Teflon-AF nanostructures. A higher magnification scan of the film revealed ordered arrays of molecular coils of Teflon-AF nanostructures implying nucleation of entangled molecular structures at the oil/water interface during the self-assembly process. This structure was not observed for (drop-cast) Teflon-AF from its solution in the solvent Novec™ (see FIG. 17) which is consistent with other published literature. The height of these structures is approximately 1.0 nm indicating a sheet width of 2 nm, implying the presence of molecular sheets of Teflon-AF (FIG. 11C).

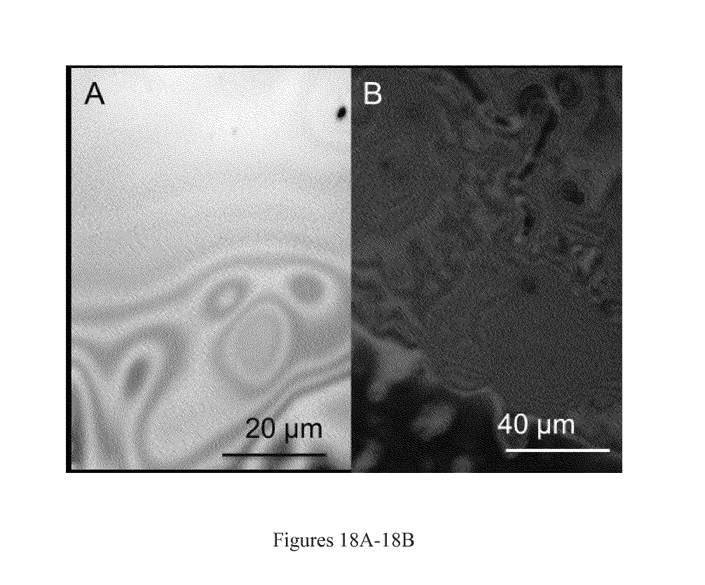

Mechanical characteristics of the Teflon-AF thin films created by an emulsion process, hereafter referred to as the Teflon-AF nanosheets, were studied by using the nanoindentation method. All the measurements were made on films, which were solution cast on a silicon substrate. To ensure intrinsic reliability, the measurements were primarily carried out on carefully selected film areas, apparently of high quality (see FIG. 18). The P-δ curves for the films were obtained by two methods, namely, constant loading/unloading rate method and the constant stiffness method (CSM).

In thin film nanoindentation studies, it is generally accepted that the depth of penetration should not exceed 30% of the film thickness in order to avoid any substrate influence. The substrate effect problem was avoided by first carrying out CSM studies on the Teflon-AF nanosheets with increasing, final depth of penetration and examining the P-δ curves for any signs of substrate effect. The data were also compared with corresponding CSM data for bare silicon substrates. The film thickness region that is free of any substrate effect was chose for the studies.

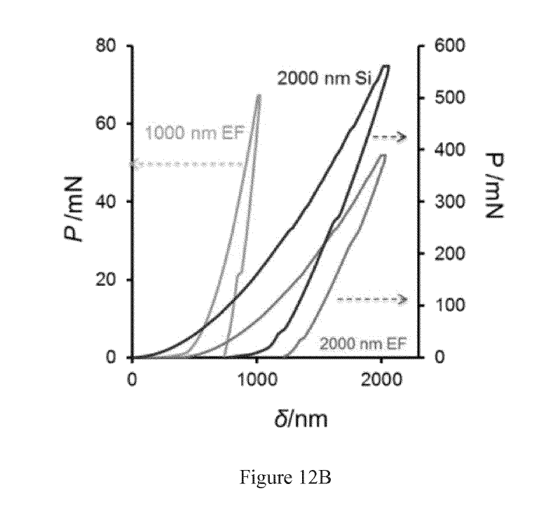

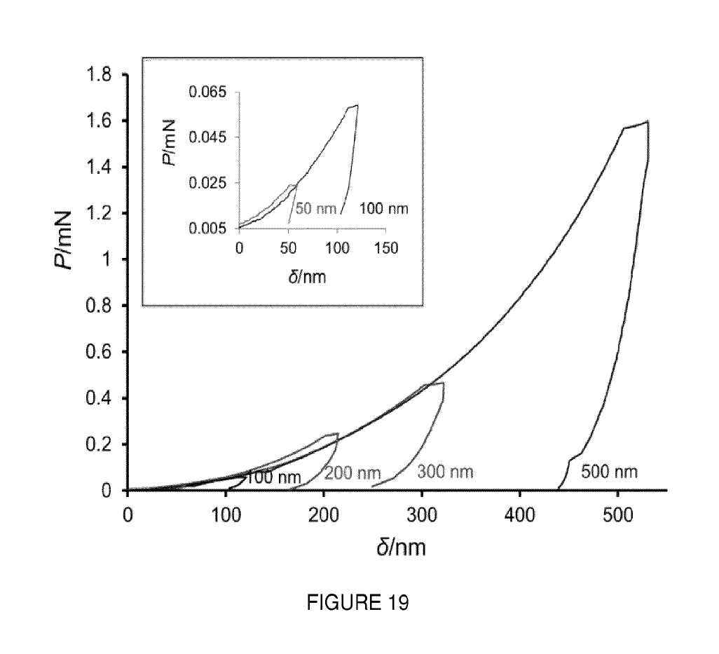

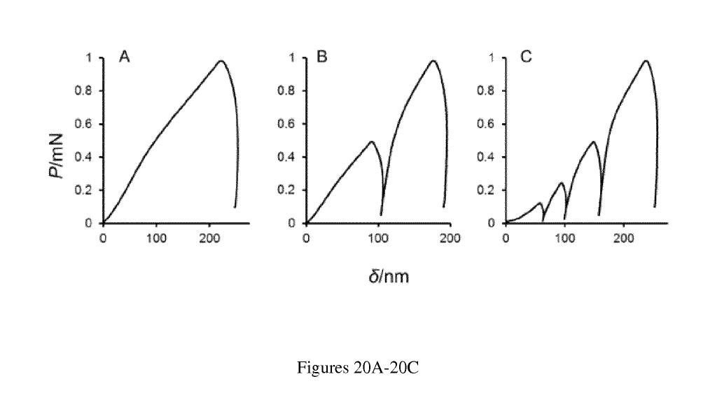

The results of CSM studies are shown in FIG. 12. In FIG. 12A, P-δ data for indention depths of 100 nm, 300 nm and 500 nm for Teflon-AF nanosheet thin films are shown. In FIG. 12B, the P-δ data for indentation depths of 1000 nm and 2000 nm for Teflon-AF nanosheet films and 2000 nm Si are reported. Unlike 100 nm and 300 nm, a characteristically typical, P-δ curve is observed for 500 nm, 1000 nm and 2000 nm Teflon-AF nanosheets films. The P-δ profile for these films appears to be qualitatively similar to that for Si and the peak load for the 2000 nm film is actually close to that for Si. It is clear that substrate effects begin to emerge above 500 nm (for an apparent film thickness of 2000 nm). A similar set of experiments were carried out for the film formed from a 0.1 wt % solution of Teflon-AF dissolved in the Novec™ solvent (see FIG. 19). Unlike the Teflon-AF nanosheets, the P-δ profiles from CSM experiments for 50 nm, 100 nm, 200 nm, 300 nm, and 500 nm indentation depths for the Novec™ film were qualitatively similar to each other and to Si, indicating a significant substrate effect even at 50 nm indentations.

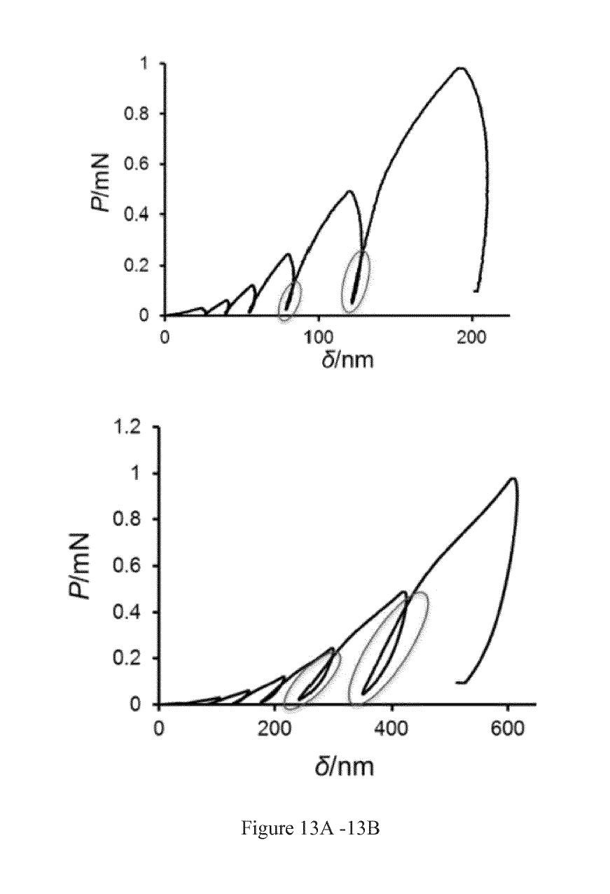

Interestingly, the unloading profiles for 100 nm and 300 nm indentation depths (FIG. 12A) also showed no elastic recovery, strongly implying that the Teflon-AF nanosheets film primarily undergoes plastic deformation. This phenomenon was investigated in a series of constant load/unload experiments where a peak load of 1 mN was reached in 1, 2, 4, and 6 steps. For comparison purposes, similar experiments were carried out for the film cast from the Novec™ solution. The Teflon-AF nanosheets results shown in FIG. 13A confirmed no elastic recovery during unloading in any of these experiments. In contrast, the films cast from the Novec™ solution showed a typical unloading profile, with significant elastic recovery (FIG. 13B). This result leads to the interesting conclusion that while Teflon-AF as a material could undergo elastic deformation, it does not, when structured as stacks of nanosheets by the emulsion process.

For the Teflon-AF nanosheets film, the P-δ profiles for the 100 nm and 300 nm indentations are not only qualitatively very different from that observed for 500 nm but the peak load for the 100 nm (70 mN) and 300 nm (74 mN) indentations are significantly lower than that for 500 nm (400 mN). More importantly, the 100 nm and 300 nm indentation P-δ curves also show significant “high” frequency perturbations (FIG. 12, highlighted by black circles) in the loading profile, which are usually associated with dislocation activity in crystals due to plastic deformations. Amorphous materials, such as this polymer film, by definition, do not have dislocations. It is believed that these high frequency perturbations are the response from the low-friction, nanosheets sliding against each other, like in a deck of cards, to dissipate the load applied to them. Since the rate of increase of applied load and the rate of dissipation by the sliding sheets are unbalanced, the tendency would be for the sheets to overreact to the applied load and then wait. This process would repeat itself in a cyclical fashion, which is what was observed.

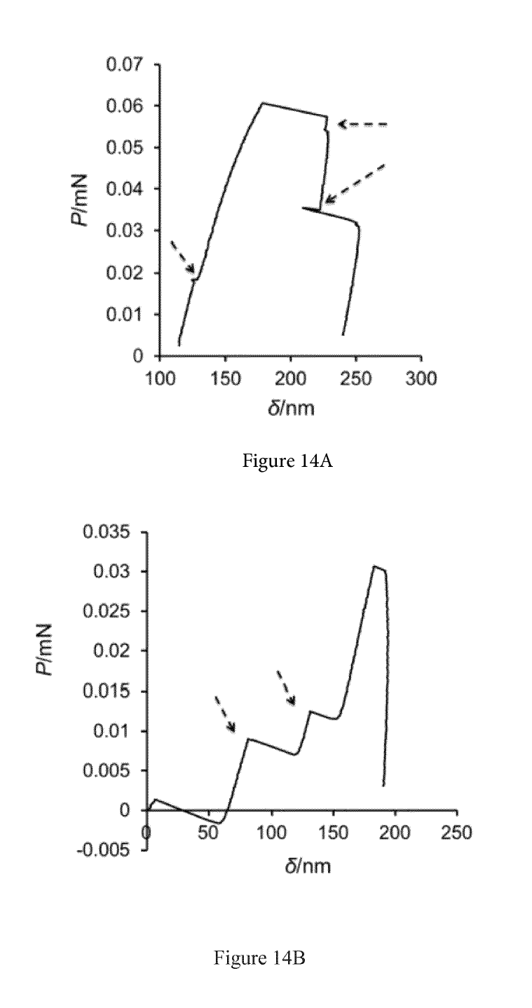

It is also very likely that for any given loading rate profile, over a period of time, the sliding sheets are unable to effectively dissipate the load, leading to an accumulation of energy in the film and creation of “shear banding” (pop-in) events such as that observed in the P-δ profiles for 100 nm and 300 nm indentations (highlighted by the blue circles in FIG. 12A). These are low frequency events. The high frequency perturbations continue to occur during these bigger “pop-in” events, as observed in the 300 nm profile. These “pop-in” events were observed in the constant load rate experiments as well, both during loading (FIG. 14A) and during unloading (FIG. 14B). Similar “pop-in” events were observed in the nanoindentation studies on nacre and were referred to as “displacement jumps”. The mechanism for load dissipation via “tablet sliding” is well established in nacre and other biological specimens. It is believed that the “displacement jumps” are correlated to the collapse and densification of the tablet interfaces in nacre, during the tablet sliding process. It is also noted that one has to use high data acquisition rates to be able to see these “pop-ins”.

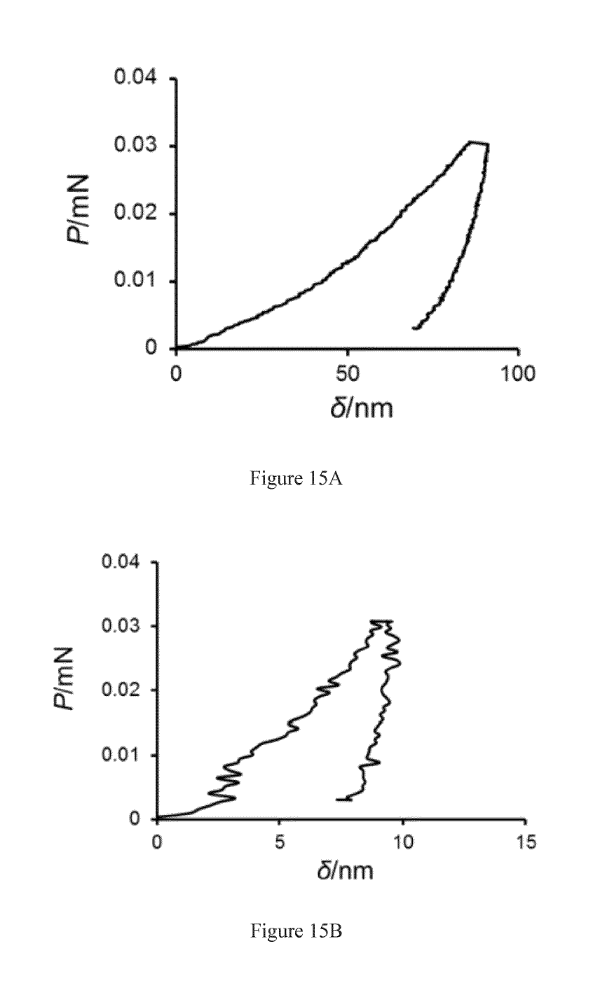

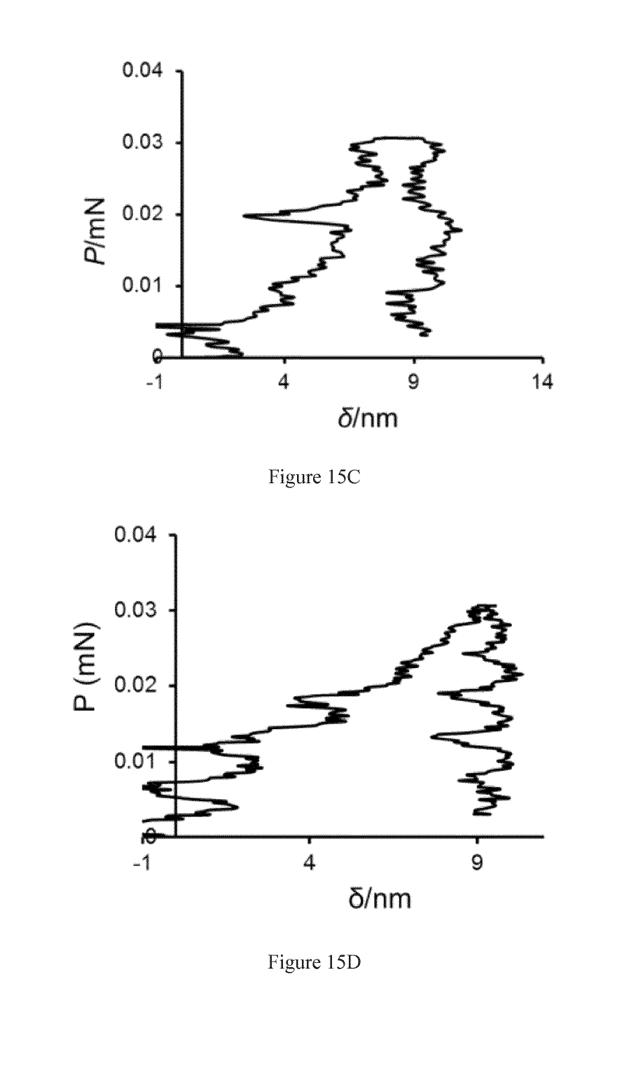

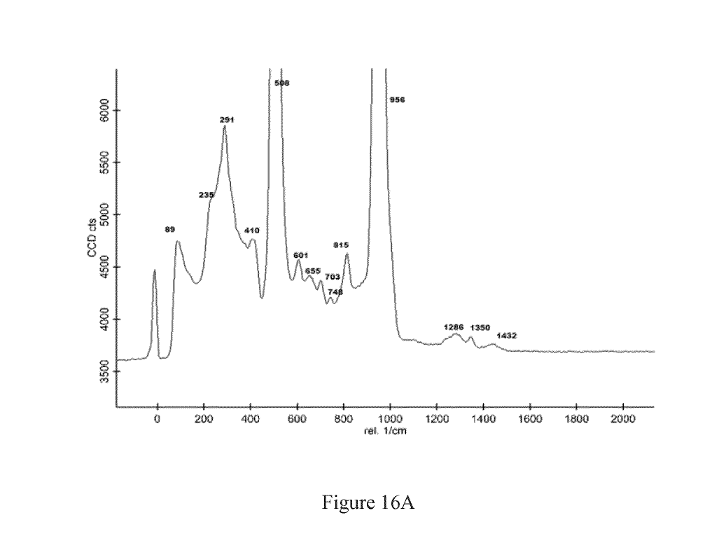

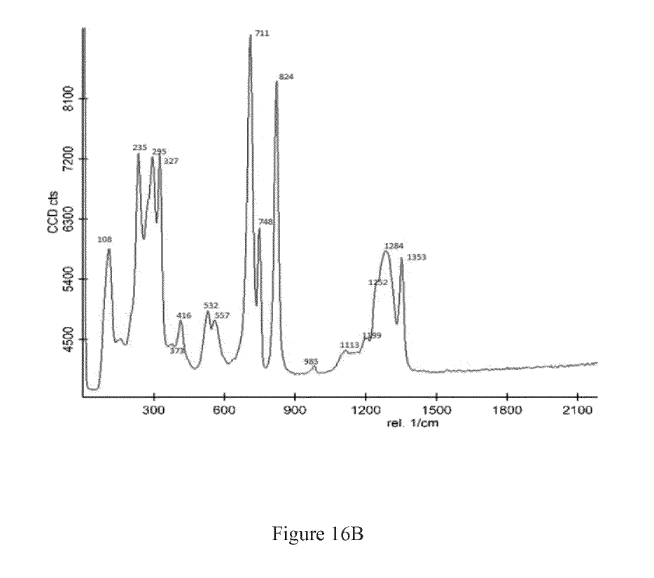

Further investigation was undertaken that Teflon-AF nanosheets film dissipates an applied load in a manner similar to the “tablets sliding” mechanism of nacre. As suggested earlier, if the high and low frequency oscillations of the P-δ profile are due to the imbalance between the applied loading rates and the sliding rates then the results of tests should support such if one is able to modulate this phenomenon of oscillations as a function of loading rates. In FIG. 15, the results are shown of the effect of loading rates on the P-δ profile, for a peak load of 30 μN. The results for Novec™ film are shown in FIG. 15A and interestingly, there are not any low or high frequency oscillations for a loading rate of 1 μN s⁻¹. The signal to noise for the P-δ profile is excellent and proves that the instrument is capable of measuring the load and displacement values very precisely, in this range. The Teflon-AF nanosheets film data are shown in FIGS. 15B, C, and D. The data in FIG. 15B, for a loading rate of 3 μN s⁻¹, clearly shows the presence of the high frequency oscillations. significant sections of the P-δ profile did not show any high frequency oscillations. In FIG. 15C, for a slower loading rate of 2 μN s⁻¹, the entire P-δ profile is populated with high frequency oscillations. Moreover, he emergence of the low frequency, “pop-in” features, both in the loading and unloading profiles is observed. As the loading rate is decreased to 1 μN s⁻¹, the high and low frequency oscillations were detected throughout the entire P-δ profile. It is interesting to note that it was possible to more clearly detect and record the oscillations as the loading rate decreased from 3 μN s⁻¹ to 1 μN s s⁻¹. This result would suggest that, in the 1 μN s⁻¹-3 μN s⁻¹ range, nano-sheets are apparently able to slide at rates that are needed to effectively dissipate the applied load. Apparently, at 3 μN s s⁻¹, a majority of the high frequency sliding was outside the range of detectability for the instrument and hence not recorded. As the loading rate was descreased to 2 μN s⁻¹ and 1 μN s⁻¹, the frequency range of “nanosheets sliding” decreased sufficiently to be within the detectability range of the instrument. This result would be consistent with the observations made by Barthelat et al. who claimed that they had to use a high data acquisition rate to observe the so-called “displacement jumps” in their nacre specimens. These results, therefore, unequivocally confirm the suggested mechanism of “sliding nanosheets” dissipating the applied load in the nanoindentation experiments. The results thus prove the creation of a physical system that mimics the “tablet sliding” nacre process.

Self-assembly of Teflon-AF nano particles on the cyclohexane/water interface resulted in the formation of stacks of mechanically robust, cohesive nano-sheets at room temperature. SEM and low angle XRD results confirm the presence of a stacked sheets structure. While XRD results suggest an approximate film thickness of 5-6 nm, AFM results strongly suggest that these nanosheets are molecular sheets of Teflon-AF. Nanoindentation studies on these nanosheets clearly demonstrated the phenomenon of “tablet sliding” that is usually associated with natural materials such as nacre. It was interesting to note the strong dependence of the sliding rate on the applied loading rate.

### 2. Ibuprofen

An implementation of the RESS process was utilized to not only produce Ibuprofen nanoparticles but able to generate drug molecule clusters, in the 1-2 nm size range, and in high yield. Although this experiment utilized Ibuprofen, it is believe the systems and methods are applicable to other compounds as well. Ibuprofen, is a widely used model compound in micronization studies. It has poor water solubility and is a non-steroidal, anti-inflammatory drug. In the last decade, several researchers have focused on supercritical CO₂ (sc-CO₂) based processes for micronization of ibuprofen nanoparticles and the smallest reported size is greater than 40 nm.

Recently, supercritical fluid based strategies have attempted by a number of investigators to prepare Ibuprofen nanoparticles. Pathak et al. produced ibuprofen nanoparticles with average size of 40±8.5 nm via modified rapid expansion of a supercritical solution into a liquid solvent (RESOLV). Hezave et al. described effect of processing conditions on the size and morphology of micronized ibuprofen particles ˜1 μm via RESS process. Recently, Bakhbakhi et al reported precipitation and compressed antisolvent (PCA) process using carbon dioxide for the production of fine particles of Ibuprofen sodium. They reported particle size ˜4 μm with narrow particle size distribution by tuning of different processing conditions. Below is described a RESS method for nanosizing ibuprofen drug into sub-10 nm nanoparticles having narrow size distribution with sufficient nanoparticle yield. In support, DLS, AFM, Raman and XRD investigations are reported for nanoparticle size, size distribution, compositional, and structural identifications of ibuprofen nanoparticles. As described below, amodified RESS process of the present invention is able to generate ibuprofen nano particles that are around 1-2 nm, essentially molecular clusters. More importantly, this process has been optimized to achieve collection yield of ˜80%, making it commercially viable.

### Experiment

Ibuprofen (pharmaceutical grade) was purchased from Sigma Aldrich Co, USA and used without further purification. Polyethyleneimine (PEI) with molecular weight of 400 kDa is used as surfactant in aqueous dispersions. High purity carbon dioxide and de-ionized water was used as the supercritical fluid medium and dispersion medium. 10 mm×10 mm dimension of well-polished mica substrates (for AFM measurements), Cu coated TEM grids (SEM studies), single crystalline quartz (for Raman measurements), p-type (100) silicon wafers (for XRD) and were considered as substrates for sample preparation.

RESS experiments were performed using an apparatus designed for nanoparticle synthesis in the temperature range from 300 K to 600 K and pressure up to 400 bar (described in Khapli, S. & Jagannathan, R. Supercritical CO₂ based processing of amorphous fluoropolymer Teflon-AF: Surfactant-free dispersions and superhydrophobic films. J Supercrit. Fluids 85, 49-56 (2014), incorporated by reference herein). In the present experiments, the chamber temperature was maintained constant at 313 K and extraction pressures was varied form 125 bar to 325 bar. These conditions were chosen to prevent undesirable particle precipitation inside the RESS apparatus and clogging at nozzle. Approximately 150 mg of ibuprofen was dissolved in supercritical CO₂ at the temperature and pressures of interest, in the range mentioned above, and the expanded solution was collected in a liquid N₂ cooled container. The collected dry ice with embedded ibuprofen nanoparticles was mixed with surfactant solutions (polyethyleneimine, PEI) at room temperature to recover the nanoparticles stabilized by surface coating of PEI.

For yield calculations, the mass of the dry ice and the ibuprofen were measured on Melter Toldeo ML104 Analytical Balance. Particle size analysis of ibuprofen nanoparticle dispersions was carried out using Malvern Zetasizer Nano ZS 90 fitted with a HeNe laser (633 nm wavelength). The scattered light signal was collected at a scattering angle of 90°. Temperature of the dispersion was maintained constant at 25° C. throughout the duration of measurement. The reported results are the average over nine measurements with the error indicating standard deviation. Atomic force microscopy was performed using Agilent 5500 Nanoscope in operating in the intermittent contact mode. Ibuprofen nanoparticles obtained from the RESS process were deposited over mica substrates for AFM characterization. Micro Raman analysis was carried out using confocal Raman microscope (WiTec alpha 300RA) using 532 nm excitation. X-ray Diffraction measurements were performed on Panalytical Empyrean X-ray diffractometer.

### Results

### Particle Size Assessment

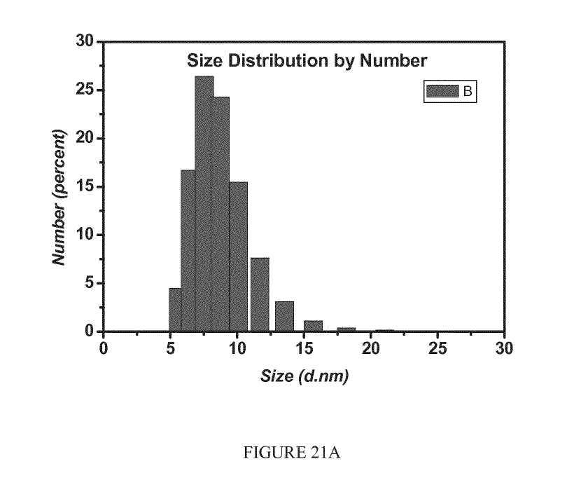

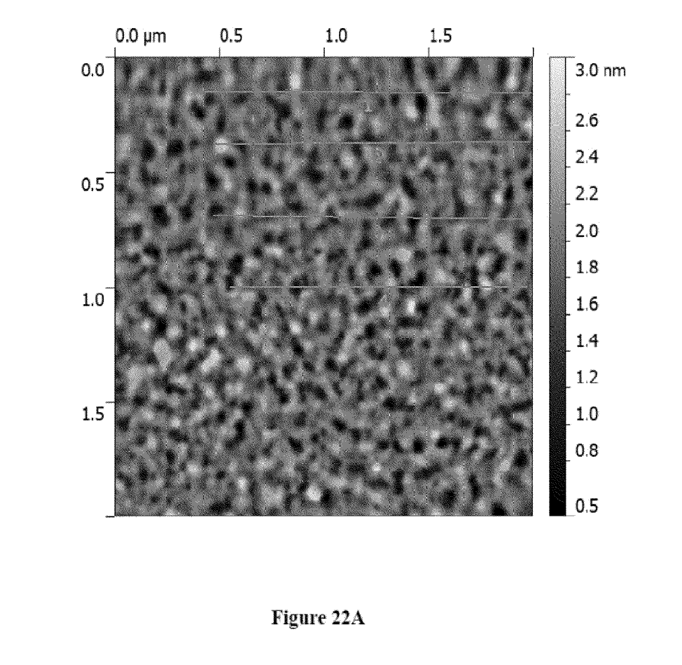

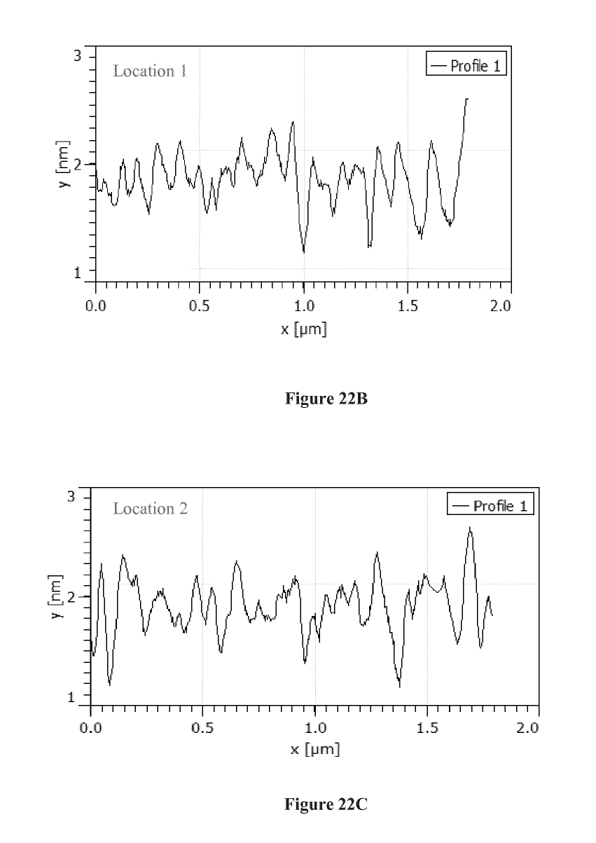

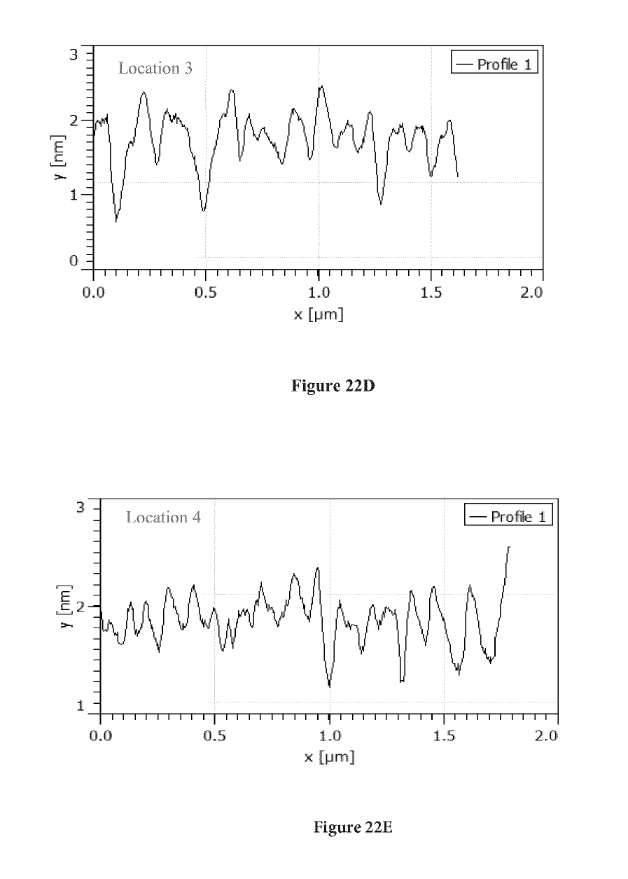

The particles generated by the described modified RESS process were primarily characterized using atomic force microscopy. FIG. 21 is a representative sample of over 25 sample slides of ibuprofen on various substrates, such as mica, silicon and quartz obtained from over ten experiments. The data is very reproducible. The average particle size of ibuprofen was found to be 2±0.5 nm. A calculation using a rigid sphere model suggests the number of clusters in a 2 nm particle to be about 3. These particles are essentially molecular clusters of ibuprofen.

The hydrodynamic size and size distribution of ibuprofen nanoparticle suspensions were measured using the technique of dynamic light scattering. FIG. 21 shows a typical number distribution profile of the RESS processed ibuprofen nanoparticle dispersions with mean diameter of 7.5 nm and size distribution width of ±3 nm (corresponding to polydispersity index ˜1.0). The smallest particle size of ibuprofen drug nanoparticles exist in literature so far is ˜40 nm. The particle sizes made using the described methods are substantially smaller, that is around 2±0.5 nm.

The difference in the average particle size measured by DLS and AFM is attributed to the swelling of drug nanoparticles in the aqueous solution. Also, DLS measures the larger hydrodynamic radius of the particle whereas AFM is a more direct, surface profilometric measurement.

### Chemical Characterization

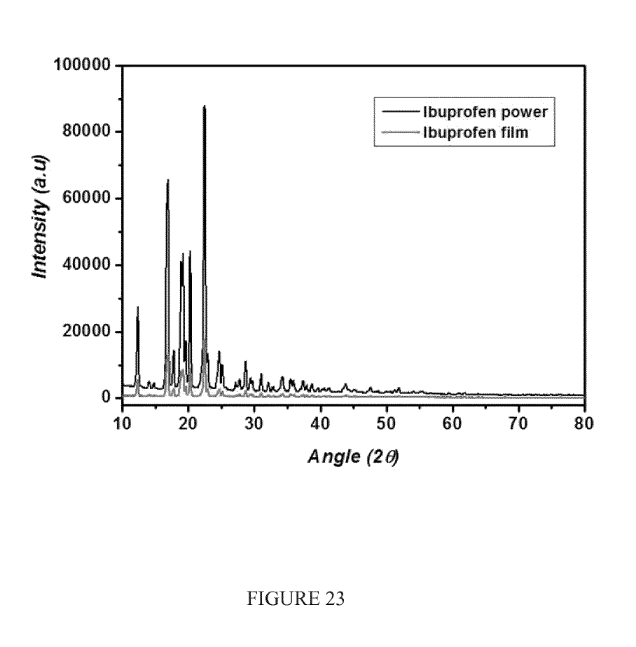

The XRD spectra recorded for unprocessed Ibuprofen powder and Ibuprofen nanoparticles generated by the system are shown in FIG. 23. Both materials exhibit a number of sharp diffraction peaks indicative of the crystalline nature of Ibuprofen. These sharp peaks in the range of 2θ˜15° to 25° are typical characteristic peaks of Ibuprofen with monoclinic P2₁/c symmetry and are observed both materials. The XRD peaks positions for the Ibuprofen nanoparticles generated by the described process are also in good agreement with those reported literature for ibuprofen powder.

### Yield Measurements

Another significant aspect of one implementation of the invention is its ability to capture the sub-10 nm Ibuprofen particles, in high yield. It is important to note that the RESS processed Ibuprofen particles are essentially molecular clusters, primarily in the size range of 2 nm and it would be not possible to efficiently capture these “gas like” molecular clusters at room temperature. In one implementation, the RESS CO₂ spray is made to pass through a “cold trap” and freeze the nanoparticles in dry ice. The percentage yield of Ibuprofen nanoparticles from the process was determined from the ratios of the nanoparticles dried/recovered (W₂) to the initial dry weight of starting material (W₁).

Percentage Yield=W₂/W₁×100

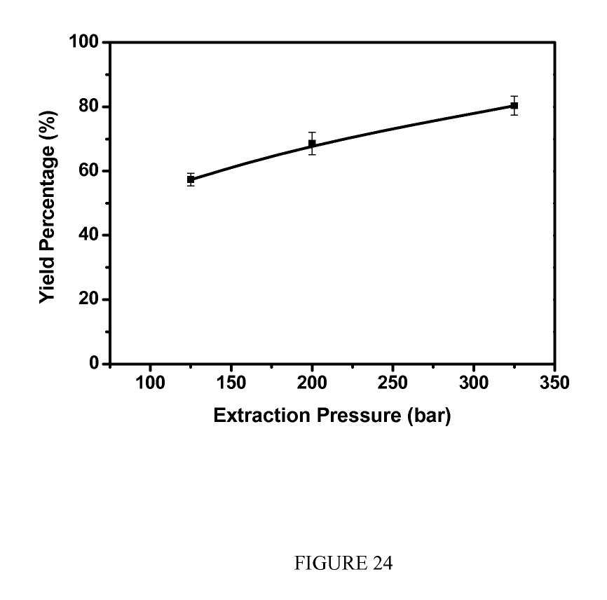

The yield was calculated for different extraction pressures and shown in FIG. 24. At extraction pressure of 325 bar, percentage yield was found to be 80±3. As shown in FIG. 24 decreasing the extraction pressure from 325 to 125 bar results in a decrease in the yield from 80% to 55%. At constant temperature, increase in extraction pressure leads to increased density and solvation strength of supercritical CO₂ leading to higher yield efficiency. One implementation of RESS collection method also takes advantage of the inherent Joule-Thomson (JT) cooling associated with the RESS expansion. The JT cooling increases with increasing extraction pressure resulting in more rapid cooling and more efficient capture of the nano-particles.

### Summary of Ibuprofen Experiment

High throughput production of sub 10 nm pharmaceutical nanoparticles of poorly water-soluble drug, ibuprofen, through a modified RESS process has been shown. AFM height profiles showed the size of nanoparticles approximately 1 nm whereas DLS measurements yield a higher, hydrodynamic size of 7.5 nm. With modified RESS technique, drug nanoparticles could be collected in high yield values, around 80% at extraction pressure of 325 bar. This method of high throughput production of sub-10 nm size drug nanoparticles is expected to enable a significant new platform for delivery of poorly water soluble drugs.

### 3. Computer Implementations

One implementation may utilize a computer system, such as shown in FIG. 6, e.g., a computer-accessible medium 620 (e.g., as described herein, a storage device such as a hard disk, floppy disk, memory stick, CD-ROM, RAM, ROM, etc., or a collection thereof) can be provided (e.g., in communication with the processing arrangement 610). The computer-accessible medium 620 may be a non-transitory computer-accessible medium. The computer-accessible medium 620 can contain executable instructions 630 thereon. In addition or alternatively, a storage arrangement 640 can be provided separately from the computer-accessible medium 620, which can provide the instructions to the processing arrangement 610 so as to configure the processing arrangement to execute certain exemplary procedures, processes and methods, as described herein, for example.

System 600 may also include a display or output device, an input device such as a keyboard, mouse, touch screen or other input device, and may be connected to additional systems via a logical network. Many of the embodiments described herein may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art can appreciate that such network computing environments can typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

Various embodiments are described in the general context of method steps, which may be implemented in one embodiment by a program product including computer-executable instructions, such as program code, executed by computers in networked environments. Generally, program modules include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps.

Software and web implementations of the present invention could be accomplished with standard programming techniques with rule based logic and other logic to accomplish the various database searching steps, correlation steps, comparison steps and decision steps. It should also be noted that the words “component” and “module,” as used herein and in the claims, are intended to encompass implementations using one or more lines of software code, and/or hardware implementations, and/or equipment for receiving manual inputs.

With respect to the use of substantially any plural and/or singular terms herein, those having skill in the art can translate from the plural to the singular and/or from the singular to the plural as is appropriate to the context and/or application. The various singular/plural permutations may be expressly set forth herein for the sake of clarity.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A process comprising:
expanding a supercritical solution of a compound of interest through a capillary at supersonic speeds;
creating a stream of nanoparticles dispersed in a gaseous medium;
cooling the stream of nanoparticles below the freezing point of the gaseous dispersion medium at least in part via application of external cooling provided by liquid coolant; and
capturing the nanoparticles in a solid matrix via a gas-to-solid phase change.

2. The process of claim 1, wherein the cooling further comprises utilizing local cooling in the stream due to the Joule-Thomson effect.

3. The process of claim 1, wherein the liquid coolant is liquid nitrogen.

4. The process according to claim 1, wherein the supercritical solution comprises the compound of interest, a supercritical solvent, and a solid co-solvent.

5. The process according to claim 4, wherein the sublimation point of the supercritical solvent comprises CO₂ at atmospheric pressure is above 77K.

6. The process according to claim 1, wherein the supercritical solution comprises no solid co-solvent.

7. The process according to claim 1, wherein greater than 80% of the nanoparticles with diameter between about 1-100 nm are captured.

8. The process according to claim 1, wherein greater than 90% of the nanoparticles with diameter between about 1-100 nm are captured.

9. The process according to claim 1 wherein the captured nanoparticles have an average size of less than 50 nm.

10. The process according to claim 1 wherein the captured nanoparticles have an average size of less than 20 nm.

11. The process according to claim 1 wherein the captured nanoparticles have an average size of less than 10 nm.

12. The process according to claim 11, wherein the captured nanoparticles have an average size of about 2 nm.

13. The process according to claim 1, wherein greater than 80% of the nanoparticles with diameter between about 2-100 nm are captured.

14. The process according to claim 1, wherein the cooling of the jet of ultra-fine particles occurs at or below pressure of 300 bar.

15. The process of claim 1 wherein capturing the nanoparticles comprises the formation of solid carbon dioxide on the nanoparticles.

16. A nontransitory computer-readable memory having instructions thereon, the instructions comprising:
instructions for controlling expansion of a supercritical solution of a compound of interest through a jet of nanoparticles particles dispersed in a gas;
instructions for cooling the jet of ultra-fine particles below the sublimation point of the gaseous dispersion medium at least in part via application of external cooling provided by liquid coolant; and
wherein the nanoparticles are separated are captured in a solid matrix via a gas-to-solid phase change.

17. The nontransitory computer-readable memory of claim 16, wherein the cooling further comprises utilizing the local cooling in the jet due to the Joule-Thomson effect.

18. The nontransitory computer-readable memory of claim 16, wherein the liquid coolant is liquid nitrogen.

19. The nontransitory computer-readable memory according to claim 16, wherein gas comprises CO₂ and the sublimation point of a supercritical solvent comprises CO₂ at atmospheric pressure is above 77K.

20. The nontransitory computer-readable memory according to claim 16, wherein greater than 90% of the nanoparticles particles with diameter between about 5-100 nm are captured.

21. The nontransitory computer-readable memory according to claim 16, wherein the cooling of the jet of nanoparticles particles occurs at or below pressure of 300 bar.

22. The nontransitory computer-readable memory of claim 16 wherein capturing the nanoparticles comprises the formation of solid carbon dioxide on the nanoparticles particles.

23. A system for collecting nanopoarticles comprising:
a feed system for providing compound of interest;
a feed cooling system having a pump, a cooling bath, and a heat exchange, the feed cooling system providing a supercritical solution of the compound of interest to a high pressure vessel;
the high-pressure vessel, which is divided by a piston head, into a formulation chamber and a control chamber; and
a coolant vessel in communication with the high-pressure vessel through a capillary tube.

24. The system of claim 23, wherein the formulation chamber comprises a stirrer.

25. The system of claim 23, wherein the he formulation chamber further includes one or more viewing windows.

26. The system of claim 23, wherein the formulation chamber comprises a heater.

27. The system of claim 23, wherein the piston is a gas-tight, floating piston head.
